(19) United States
(12) Patent Application Publication
(10) Pub. No.: US 2008/0004560 A1
(43) Pub. Date: Jan. 3, 2008

(54) Flexible Bag Wrap Adapted For Use In An Incontinence Management System

(76) Inventor: Mark Miskie, Charotte, NC (US)

Correspondence Address: SCHWARTZ LAW FIRM, P.C., 6100 FAIRVIEW ROAD, SUITE 11350, CHARLOTTE, NC, 28210, US

(21) Appl. No.: 11/667,651

(22) PCT Filed: Nov. 12, 2004
(86) PCT No.: PCT/US04/38491

§ 371 Date: May 11, 2007

(51) Int. Cl. A61F 13/00; A61L 15/00; A61M 1/00

(52) U.S. Cl. 602/75; 602/62; 604/327

(57) ABSTRACT

A flexible bag wrap is adapted for use in an incontinence management system. The bag wrap includes a flexible base adapted for wrapping around a body part of a user. The base includes a three-dimensional fabric spacer. The fabric spacer has an inside facing for residing nearest the skin of the user, an outside facing opposite the inside facing, and an intermediate spacer yarn interconnecting the inside and outside facings. A collection bag is carried by the base adjacent the outside facing of the fabric spacer, and is adapted for collecting urine excreted by the user.

### TECHNICAL FIELD AND BACKGROUND OF THE INVENTION

This invention relates generally to incontinence management, and more specifically, to a flexible bag wrap adapted for being secured to the leg of a user for collecting excreted urine. The invention is especially useful for controlling multiple instances of leakage.

An estimated 19 million North American adults suffer urinary incontinence with severity ranging from partial to complete loss of bladder control. They may experience varying degrees of urine loss, and the incontinence may change over time. For example, some adults with light incontinence may leak a little when they laugh or cough, while others with heavy incontinence may be experience continuous leakage. No two cases are alike, and no two adults are affected by incontinence the same way.

Incontinence is not an inevitable part of aging, and it is not a disease. In fact, in most cases, it is merely a symptom or side-effect of another medical condition. Male urinary incontinence may be caused by any number of health conditions including prostate surgery, neurologic disease or injury (Parkinson's disease, stroke or spinal cord injury), obstructed urination, and certain birth defects or chronic medical conditions such as diabetes. In many cases, incontinence can be cured and it can always be managed. Many women will experience periods of urinary incontinence caused by childbirth and menopause.

Presently available commercial products address all levels of urinary incontinence. Existing leg bags are typically secured to the body using one or more narrow latex straps. These straps reduce circulation to the lower leg, are generally uncomfortable, and can quickly break down the skin causing open sores and infection.

### SUMMARY OF INVENTION

Therefore, it is an object of the invention to provide a flexible bag wrap adapted for use in an incontinence management system.

It is another object of the invention to provide a flexible bag wrap designed for being secured to any body part including the lower leg, thigh, and waist.

It is another object of the invention to provide a flexible bag wrap which is convenient to use and comfortable to wear.

It is another object of the invention to provide a flexible bag wrap which incorporates a fabric spacer that maximizes breathability, insulation, compression, recovery, and durability.

It is another object of the invention to provide a flexible bag wrap which incorporates a spacer fabric that can combine as many as three different fibers in any one desired construction, exploiting the particular properties of each fiber in the two facings and the spacer yarns between them.

It is another object of the invention to provide a flexible bag wrap which incorporates a spacer fabric constructed using all homogenous materials and no glues or laminates.

It is another object of the invention to provide a flexible bag wrap which is launderable.

It is another object of the invention to provide a flexible bag wrap which incorporates a spacer that retains its qualities even after repeated washings.

It is another object of the invention to provide a flexible bag wrap which incorporates a fabric spacer that keeps the skin (which contacts the wrap) dry, and prevents growth of fungus and bacteria.

It is another object of the invention to provide a flexible bag wrap which is relatively light.

It is another object of the invention to provide a flexible bag wrap which increases the distribution of pressure against the lower leg of the user.

It is another object of the invention to provide a flexible bag wrap which resists slipping on the lower leg of the user.

It is another object of the invention to provide a flexible bag wrap which is adjustable to fit all users.

It is another object of the invention to provide a flexible bag wrap which includes an integrally formed collection bag.

It is another object of the invention to provide a flexible bag wrap which includes a removable and disposable collection bag.

These and other objects of the present invention are achieved in the preferred embodiments disclosed below by providing a flexible bag wrap adapted for use in an incontinence management system. The bag wrap includes a flexible base adapted for wrapping around a body part of a user. The base includes a three-dimensional fabric spacer. The fabric spacer has an inside facing for residing nearest the skin of the user, an outside facing opposite the inside facing, and an intermediate spacer yarn interconnecting the inside and outside facings. A collection bag is carried by the base adjacent the outside facing of the fabric spacer and is adapted for collecting bodily fluid, such as urine, excreted by the user.

According to another preferred embodiment of the invention, the base has a central body portion and opposing attachment wings.

According to another preferred embodiment of the invention, the body portion and attachment wings of the base define respective top and bottom edges. At least one of the top and bottom edges of at least one of the attachment wings extends at an angle relative to an adjacent top and bottom edge of the body portion, whereby the base is ergonomically designed to fit the body part of the user.

According to another preferred embodiment of the invention, the attachment wings of the base include releasable fasteners.

According to another preferred embodiment of the invention, the inside facing of the fabric spacer has a friction surface.

According to another preferred embodiment of the invention, the friction surface comprises a plurality of longitudinal friction strips.

According to another preferred embodiment of the invention, the spacer has a loft greater than 0.06 inches.

According to another preferred embodiment of the invention, the outside facing of the spacer comprises a fine mesh fabric having at least 400 uniform, mesh openings per square inch.

According to another preferred embodiment of the invention, the outside facing of the spacer comprises moisture wicking fibers adapted for promoting moisture flow by capillary wicking. The moisture wicking fibers are selected from a group consisting of polyester, nylon, and acrylic.

According to another preferred embodiment of the invention, the inside facing of the spacer comprises an open mesh fabric having less than 50 uniform, mesh openings per square inch.

According to another preferred embodiment of the invention, the inside facing of the spacer comprises moisture-absorbent fibers adapted for drawing moisture from the inside facing of the spacer through the spacer yarn. The moisture-absorbent fibers are selected from a group consisting of cotton, wool, and hydrophilic polyester.

According to another preferred embodiment of the invention, the intermediate spacer yarn of the spacer comprises moisture wicking fibers adapted for promoting moisture flow from the inside facing to the outside facing by capillary wicking. The moisture wicking fibers are selected from a group consisting of polyester, nylon, and acrylic.

In yet another embodiment, the invention is a flexible bag wrap adapted for use in an incontinence management system. The bag wrap has a flexible base adapted for wrapping around a body part of a user. The base has an inside facing adapted for residing nearest the skin, and an outside facing opposite the inside facing. A collection bag is integrally and permanently formed with the base, and is adapted for collecting bodily fluid, such as urine, excreted by the user. The terms “integrally and permanently formed” mean that the bag and base are inseparably joined together as a single unit. The means for joining may include sewing, riveting, gluing, heat welding, or other suitable permanent attachment means.

In yet another embodiment, the invention is a flexible bag wrap adapted for use in an incontinence management system. The bag wrap includes a flexible base for wrapping around a body part of a user. The base comprises a three-dimensional fabric spacer having an inside facing adapted for residing nearest the skin, an outside facing opposite the inside facing, and an intermediate spacer yarn interconnecting the inside and outside facings.

### BRIEF DESCRIPTION OF THE DRAWINGS

Some of the objects of the invention have been set forth above. Other objects and advantages of the invention will appear as the description proceeds when taken in conjunction with the following drawings, in which:

FIG. 1 is an environmental view of the flexible bag wrap according to one preferred embodiment of the present invention, and showing the bag wrap secured to the lower leg of a user;

FIG. 2 is a front side of the bag wrap;

FIG. 3 is a back side of the bag wrap;

FIG. 4 is a side view of the bag wrap;

FIG. 5 is an enlarged, fragmentary cross-sectional view of the fabric spacer; and

FIG. 6 is an environmental view of the flexible bag wrap according to a second preferred embodiment of the present invention.

### DESCRIPTION OF THE PREFERRED EMBODIMENT AND BEST MODE

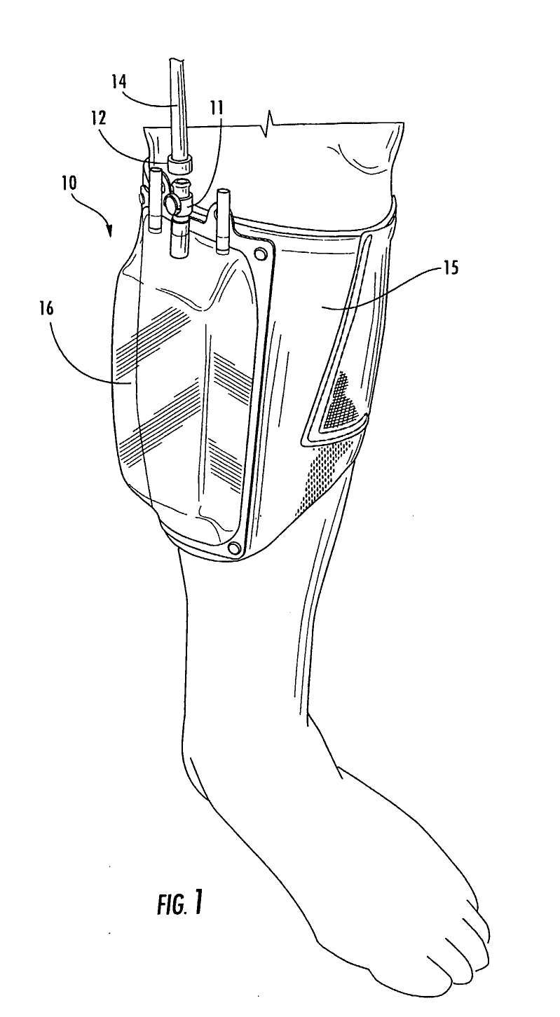

Referring now specifically to the drawings, a flexible bag wrap according to the present invention is illustrated in FIG. 1, and shown generally at reference numeral 10. The bag wrap 10 is applicable for use in an incontinence management system, such as disclosed in Applicant's published International (PCT) Application Nos. PCT/US2002/039278, PCT/US2003/027060, PCT/US2002/034541. The complete disclosure of these applications is incorporated herein by reference. More specifically, the bag wrap 10 is especially useful for an individual confined to a wheel chair. The bag wrap 10 includes means for convenient attachment and removal to and from the lower leg, and a standard quick connect/quick release fitting 11 for attaching a complementary terminal end 12 of a fluid transfer tube 14.

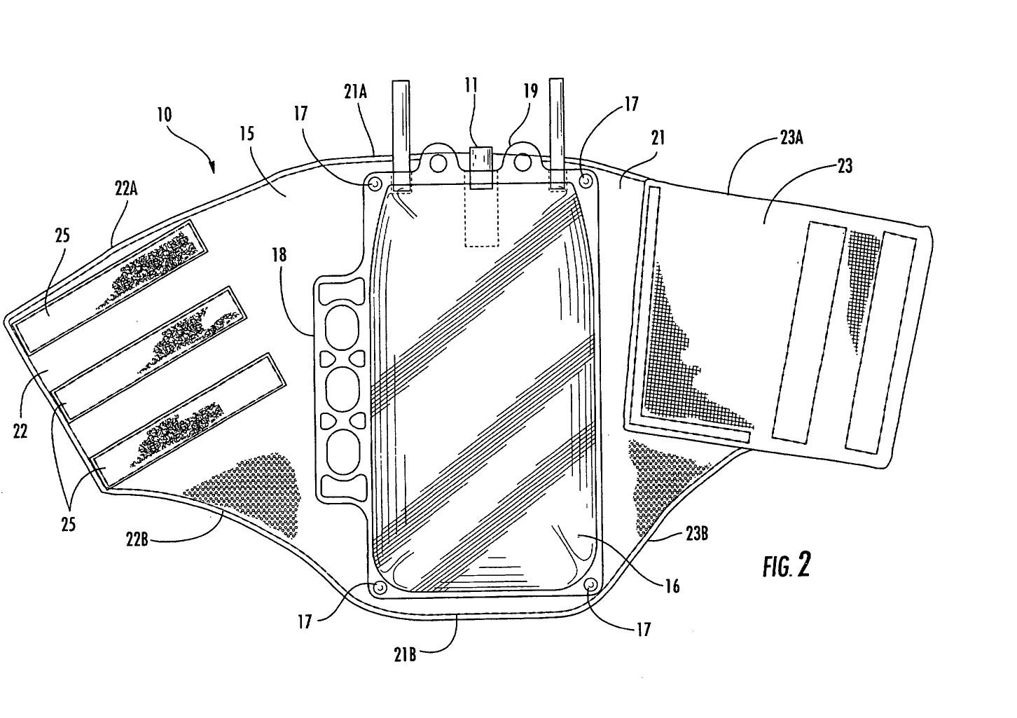

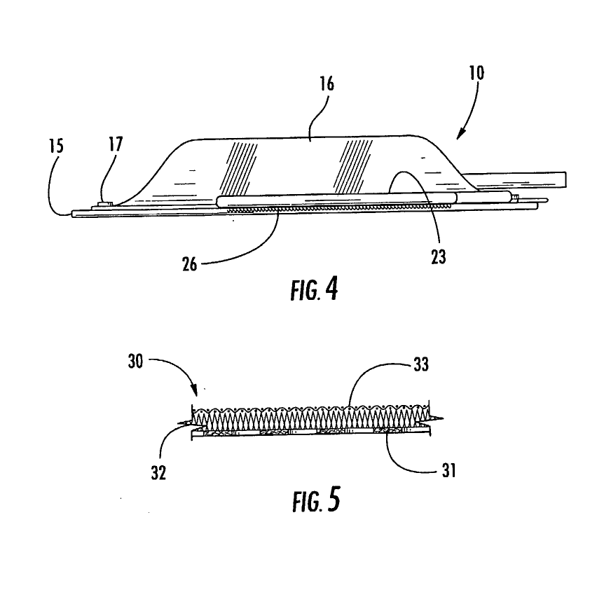

As best shown in FIGS. 2, 3, and 4, the bag wrap 10 comprises a flexible base 15 designed to extend around the leg of the user, and a thermoplastic vacuum-formed collection bag 16 for receiving and collecting excreted urine. According to one embodiment, the collection bag 16 is permanently and integrally formed with the base 15. The bag attachment means may comprise rivets 17 or other means, such as sewing, gluing, or heat welding. The vacuum-formed bag 16 has a generally flat back that remains flat when the bag is filled to capacity, as shown in FIG. 4. The inlet fitting 11 with an integrated one-way valve is located at the top of the bag 16 for connecting the fluid transfer tube 14. A vertical drain port and air vent are also provided for conveniently emptying the bag 16 when full. An integrally-formed or removable clamp (not shown) operates to releasibly close the drain port when the bag wrap 10 is in use. To facilitate handling, the bag 16 may further include side and top handles 18 and 19 with respective finger openings.

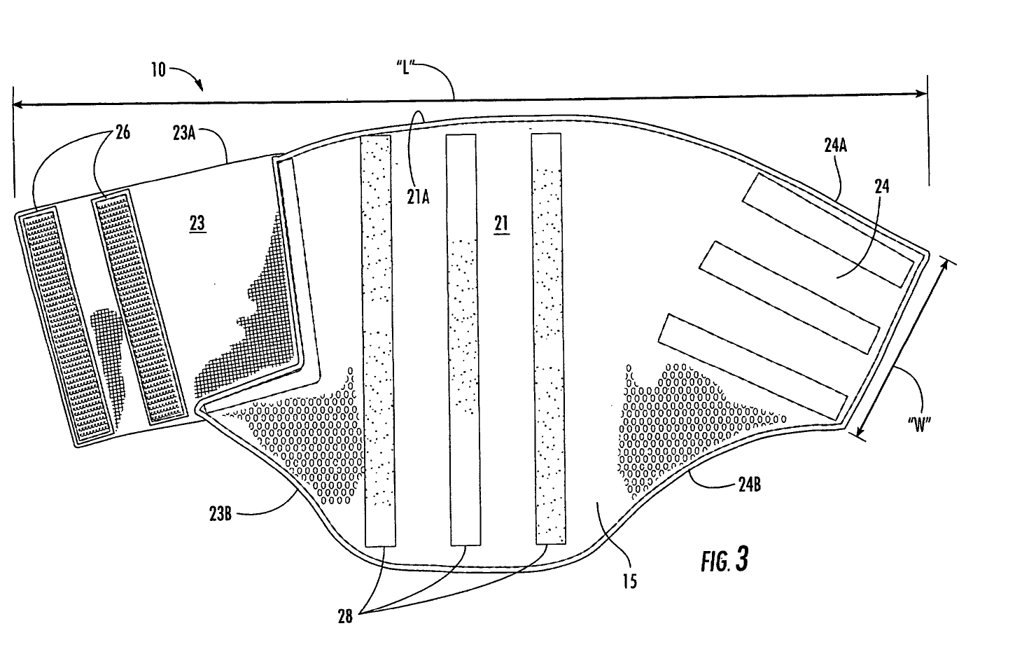

Referring to FIGS. 2 and 3, the flexible base 15 defines a central body portion 21 and opposing attachment wings 22 and 23—each having top and bottom edges. The top and bottom edges 21A, 21B of the body portion 21 are generally horizontal, whereas the top edges 22A, 23A of the attachment wings 22, 23 extend outwardly at a slight angle relative to the top edge 21A. The angle of the top edge 22A relative to the edge 21A is preferably between 15 and 30 degrees. The angle of the top edge 23A relative to the edge 21A is preferably between 10 and 30 degrees. The bottom edges 22B, 23B of the attachment wings 22, 23 are preferably contoured in a generally concave design relative to the bottom edge 21B.

Longitudinal strips 25 of loop material are located on the outside major surface of the flexible base 15, and are designed to mate with complementary hooks formed in lateral strips 26 located on the inside major surface of the flexible base 15. When wrapped around the leg in an area of the calf, as shown in FIG. 1, the flexible base 15 provides a comfortable, ergonomic and secure fit. The attachment wings 22, 23 have a minimum width dimension “W” which is preferably greater than 20 percent of the overall length “L” of the base 15. Consequently, any pressure applied to the lower leg is distributed over a greater surface area, as compared to more traditional strap attachments. For added support on the leg, the inside surface of the base 15 within the body portion 21 may include one or more longitudinal friction strips 28 extending substantially from the top edge 21A to the bottom edge 21B. The friction strips 28 are formed of any high friction material, such as rubber, which resists sliding against the skin.

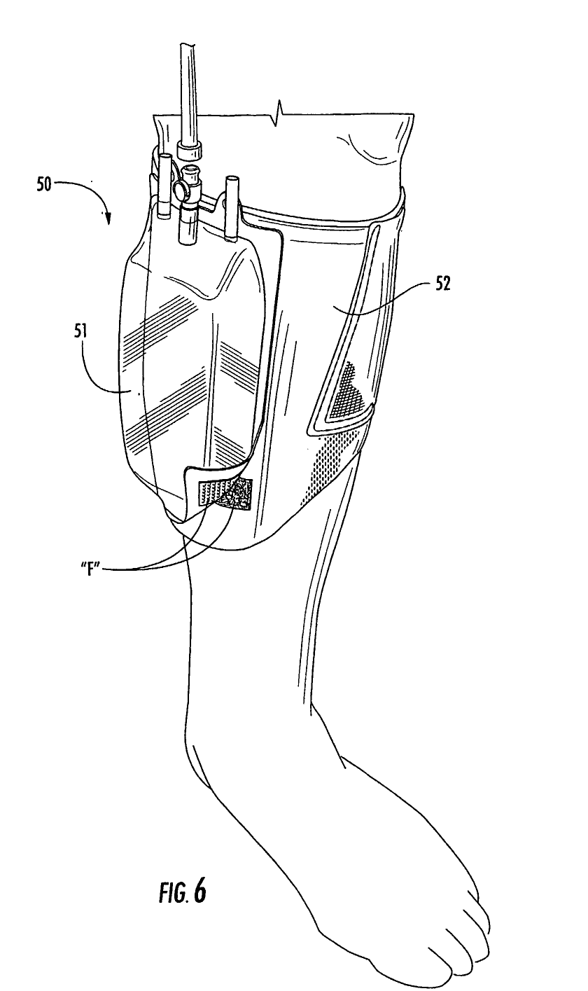

The preferred material construction of the flexible base 15 is illustrated in FIG. 5. The base 15 comprises a three-dimensional spacer fabric 30 consisting of two independently constructed fabric facings 31 and 32 integrally formed with a system of intermediate spacer yarns 33. Examples of this construction are described in U.S. Pat. Nos. 6,103,641 and 6,627,562 incorporated herein by reference. The spacer 30 is produced on a two needle bar Raschel machine, and has a loft ranging from 0.06 inches to more than 0.6 inches. According to one embodiment, the loft is approximately 0.1875 inches.

The inside facing 31 which resides nearest the skin of the user comprises a highly breathable, open mesh fabric which promotes air circulation within and through the spacer 30. Preferably, the mesh fabric has less than 50 uniform, generally oval, mesh openings per square inch. This fabric consists substantially of low moisture-absorbent fibers, such as acrylic, or non-absorbent hydrophobic fibers, such as polypropylene. These fibers resist swelling and compression when wet, and promote the rapid transport of moisture away from the inner layer of the spacer 30 and the skin of the user. The quantity of low and/or non-absorbent fibers is greater than 50% of the total fiber incorporated in the facing 31, and more preferably, between 85% and 100%. A “low absorbent” fiber is defined as one with less than 10% swell when wet. A “non-absorbent” fiber has less than 5% swell when wet. To deter the growth of fungus and bacteria, the inside facing may also include antimicrobial fibers comprising silver. Suitable antimicrobial fibers are disclosed in prior U.S. Pat. No. 6,723,428 incorporated herein by reference.

The spacer yarns 33 comprise moisture-wicking fibers, such as acrylic and polyester, which are made to promote the transport of moisture by capillary wicking. Examples of commercially available wicking fibers include Isolfil® by Di Borio Fiorino, Dacron® by E. I. du Pont de Nemours and Company, and Coolmax® by Invista. The mechanical structure and compressibility of the fiber determine its overall wicking potential. CoolMax® fibers, for example, have four longitudinal channels built into their cross-sectional geometry giving a 20% higher perimeter area than traditional round fibers. The result is higher water/vapor transport through enhanced surface exposure for capillary action. Capillary action attracts water from the inside facing 31 of the spacer 30 where it is abundant and rapidly transports it to the outside facing 32 of the spacer 30 where it is less abundant. Preferably, the spacer yarns 33 consist of between 85% and 100% wicking fibers.

The outside fabric facing 32 adjacent the spacer yarns 33 is constructed of high moisture-absorbent fibers, such as cotton, wool, and hydrophilic polyester, and/or wicking fibers, such as polyester and acrylic, which operate to draw moisture outwardly from the inside facing 31 and spacer yarns 33, and disperse it over the entire surface area of the outside facing 32. The outside facing 32 comprises a relatively fine mesh fabric having at least 400 uniform, generally round, mesh openings per square inch. A “high” moisture-absorbent fiber is defined herein as one which swells in excess of 20% when wet.

In an alternative embodiment shown in FIG. 6, the bag wrap 50 incorporates a removable and disposable collection bag 51. The collection bag 51 is removably attached to the flexible base 52 using complementary fasteners “F”, such as hook and loop. The bag 51 of this embodiment is completely removable and exchangeable. All other elements and features of the bag wrap 50 remain identical to that described above. The flexible bag wrap 50 may therefore comprise both the flexible base 52 and collection bag 51 in combination, or only the flexible base 52 constructed of the spacer fabric previously described. For added elasticity, the three-dimensional spacer fabric may further include elastic yarns, such as spandex. Alternatively, the attachment wings may be sewn directly to the fabric spacer, and may be formed of a different fabric incorporating elastic yarns.

A flexible bag wrap is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. A flexible bag wrap adapted for use in an incontinence management system, said bag wrap comprising:
(a) a flexible base adapted for wrapping around a body part of a user, and comprising a three-dimensional fabric spacer, said fabric spacer having an inside facing for residing nearest the skin of the user, an outside facing opposite the inside facing, and an intermediate spacer yarn interconnecting the inside and outside facings; and
(b) a collection bag carried by said base adjacent the outside facing of said fabric spacer, and adapted for collecting bodily fluid.

2. A flexible bag wrap according to claim 1, wherein said base comprises a central body portion and opposing attachment wings.

3. A flexible bag wrap according to claim 2, wherein the body portion and attachment wings of said base define respective top and bottom edges, and at least one of the top and bottom edges of at least one of the attachment wings extends at an angle relative to an adjacent top and bottom edge of the body portion, whereby said base is ergonomically designed to fit the body part of the user.

4. A flexible bag wrap according to claim 2, wherein the attachment wings of said base comprise releasable fasteners.

5. A flexible bag wrap according to claim 2, wherein the inside facing of said fabric spacer comprises a friction surface.

6. A flexible bag wrap according to claim 5, wherein the friction surface comprises a plurality of longitudinal friction strips.

7. A flexible bag wrap according to claim 1, wherein said fabric spacer has a loft greater than 0.06 inches.

8. A flexible bag wrap according to claim 1, wherein the inside facing of said spacer comprises an open mesh fabric having less than 50 uniform, mesh openings per square inch.

9. A flexible bag wrap composite according to claim 8, wherein the inside facing of said spacer comprises moisture-absorbent fibers adapted for drawing moisture from the inside facing of said spacer through said spacer yarn, said moisture-absorbent fibers being selected from a group consisting of cotton, wool, and hydrophilic polyester.

10. A flexible bag wrap according to claim 1, wherein the outside facing of said spacer comprises a fine mesh fabric having at least 400 uniform, mesh openings per square inch.

11. A flexible bag wrap according to claim 10, wherein the outside facing of said spacer comprises moisture wicking fibers adapted for promoting moisture flow by capillary wicking, said moisture wicking fibers being selected from a group consisting of polyester, nylon, and acrylic.

12. A flexible bag wrap according to claim 1, wherein the intermediate spacer yarn of said spacer comprises moisture wicking fibers adapted for promoting moisture flow from the inside facing to the outside facing by capillary wicking, said moisture wicking fibers being selected from a group consisting of polyester, nylon, and acrylic.

13. A flexible bag wrap adapted for use in an incontinence management system, comprising:
(a) a flexible base adapted for wrapping around a body part of a user, and having an inside facing adapted for residing nearest the skin, an outside facing opposite the inside facing; and
(b) a collection bag integrally and permanently formed with said base, and adapted for collecting bodily fluid.

14. A flexible bag wrap according to claim 13, wherein said base comprises a central body portion and opposing attachment wings.

15. A flexible bag wrap according to claim 14, wherein the body portion and attachment wings of said base define respective top and bottom edges, and at least one of the top and bottom edges of at least one of the attachment wings extends at an angle relative to an adjacent top and bottom edge of the body portion, whereby said base is ergonomically designed to fit the body part of the user.

16. A flexible bag wrap according to claim 14, wherein the attachment wings of said base comprise releasable fasteners.

17. A flexible bag wrap according to claim 13, wherein the inside facing of said base comprises a friction surface.

18. A flexible bag wrap according to claim 17, wherein the friction surface comprises a plurality of longitudinal friction strips.

19. A flexible bag wrap adapted for use in an incontinence management system, said bag wrap comprising a flexible base for wrapping around a body part of a user, and including a three-dimensional fabric spacer having an inside facing adapted for residing nearest the skin, an outside facing opposite the inside facing, and an intermediate spacer yarn interconnecting the inside and outside facings.

20. A flexible bag wrap according to claim 19, wherein the inside facing of said spacer comprises an open mesh fabric having less than 50 uniform, mesh openings per square inch; and wherein the outside facing of said spacer comprises a fine mesh fabric having at least 400 uniform, mesh openings per square inch.
